(19) United States
(12) Patent Application Publication
(10) Pub. No.: US 2025/0306351 A1
(43) Pub. Date: Oct. 2, 2025

(54) TOTAL MAGNIFICATION CALCULATION OF DIGITAL MICROSCOPE HAVING DIGITAL BINOCULARS AND INTERCHANGEABLE EYEPIECES

(71) Applicant: Alcon Inc., Fribourg (CH)

(72) Inventors: Jing Xu, Irvine, CA (US); John Park, Irvine, CA (US); Yuchao Liu, Irvine, CA (US); Lingfeng Yu, Rancho Santa Margarita, CA (US)

(73) Assignee: Alcon Inc., Fribourg (CH)

(21) Appl. No.: 19/089,547

(22) Filed: Mar. 25, 2025

Related U.S. Application Data
(60) Provisional application No. 63/570,298, filed on Mar. 27, 2024.

(51) Int. Cl. G02B 21/00; G02B 21/02; G02B 21/20

(52) U.S. Cl. CPC G02B 21/0028; G02B 21/008; G02B 21/025; G02B 21/20

(57) ABSTRACT

A visualization system for use with an ophthalmic microscope having a pair of image sensors includes a first pair and multiple second pairs of interchangeable eyepieces each having a corresponding magnification level and field-of-view (FOV), and digital binoculars in communication with the image sensors. The digital binoculars include a housing configured to connect to the ophthalmic microscope and defining a housing cavity, a pair of annular bases connected to the housing configured to separately engage the first and multiple second pairs of interchangeable eyepieces to provide the FOV, and surrounding a corresponding center axis or designated position, and a pair of micro displays positioned within the housing cavity. Each respective one of the micro displays is arranged on the corresponding center axis or designated position.

### CROSS-REFERENCE TO RELATED APPLICATIONS

The present application claims the benefit of priority to U.S. Provisional Application No. 63/570,298 filed Mar. 27, 2024, which is hereby incorporated by reference in its entirety.

### INTRODUCTION

The present disclosure relates to automated systems and methods for calculating the total magnification of a visualization system.

Digital and hybrid camera-equipped microscopes are able to acquire digital pixel images of a target object. Digital images of the target object may be either magnified or reduced depending on the object field-of-view (FOV) and size of the image sensors. An ophthalmic digital microscope in particular includes three major components: (i) imaging optics, which provide two slightly different views of a patient's eye area to approximate normal human stereo vision, (ii) a pair of image sensors, e.g., complementary metal-oxide-semiconductor (CMOS) sensors with a corresponding image processing unit for converting collected images to digital images, and (iii) a display screen for presenting the digital images to an attending clinician. A heads up display (HUD) system is typically used for this purpose in an ophthalmic surgical suite. In such a system, the clinician wears polarized glasses and views projected 3D images of the patient's eye area on one or more 3D display screens. While such a system provides high-resolution views of the patient's eye area, the need for polarized glasses and viewing of the 3D display screens may be suboptimal for certain users or applications, e.g., in terms of relative clinician comfort and image display qualities.

### SUMMARY

Systems and methods are disclosed herein for use with a visualization system having an ophthalmic microscope equipped with a set of digital binoculars. Such a system may be used as an alternative to the above-summarized heads up display (HUD) system for a more natural and comfortable view of the patient's eye area. The digital binoculars are mechanically coupled to the microscope and include multiple pairs of interchangeable eyepieces. Each pair of interchangeable eyepieces has a corresponding eyepiece magnification level. Also disclosed herein is a method for calculating a total magnification level of the visualization system, including magnification levels of the microscope, the digital binoculars, and the interchangeable eyepieces. Aspects of the disclosure also pertain to provision of the interchangeable eyepieces for the digital binoculars, the availability of which allows the digital binoculars to be used with multiple different pairs of eyepieces without changing out the digital binoculars.

In a contemplated construction, miniature display screens (“micro displays”) contained within a housing of the digital binoculars are viewed through separate left and right eyepieces, which respectively correspond to a clinician's left and right eye. Each eyepiece has a front lens through which the clinician views the patient's eye area on the micro displays. Each front lens is positioned relative to a respective one of the micro displays contained within the aforementioned housing.

In particular, a visualization system for use with a microscope includes multiple pairs of interchangeable eyepieces each having a corresponding magnification level and field-of-view (FOV), and digital binoculars in communication with the image sensors of the microscope. The digital binoculars includes a housing, a pair of annular bases, and a pair of micro displays. The housing is configured to connect to the microscope, defines a housing cavity. The annular bases are connected to the housing, configured to separately engage the first and second pairs of interchangeable eyepieces, and surround a corresponding designated position, e.g., a respective center axis. The pair of micro displays is positioned within the housing cavity, each respective one of the micro displays being arranged at the corresponding position.

The visualization system in accordance with another aspect of the disclosure includes a microscope, a pair of image sensors connected to the microscope, multiple pairs (at least first and second pairs) of interchangeable eyepieces each having a corresponding magnification level and object FOV, and the digital binoculars summarized above.

The above-described features and advantages and other possible features and advantages of the present disclosure will be apparent from the following detailed description when taken in connection with the accompanying drawings.

### BRIEF DESCRIPTION OF THE DRAWINGS

FIG. 1 illustrates an exemplary surgical suite having a visualization system equipped with an ophthalmic microscope, digital binoculars, and interchangeable eye pieces, with a total magnification level of the system being determined in accordance with the present disclosure.

FIG. 2 is a perspective view illustration of a representative pair of digital binoculars with multiple pairs of interchangeable eyepieces.

FIG. 3 is a perspective view illustration of the digital binoculars of FIG. 2 with one eyepiece removed for illustrative clarity.

FIG. 4 is a side view illustration of an interchangeable eyepiece and an optional annular adapter piece for connecting the eyepiece to the digital binoculars of FIGS. 1-3.

FIGS. 5A and 5B illustrate a moveable platform for use with the interchangeable eyepieces disclosed herein.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

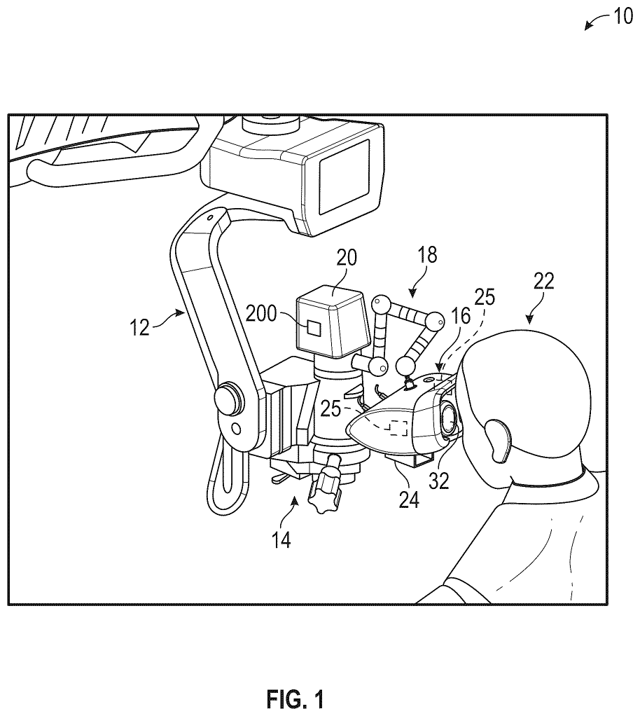

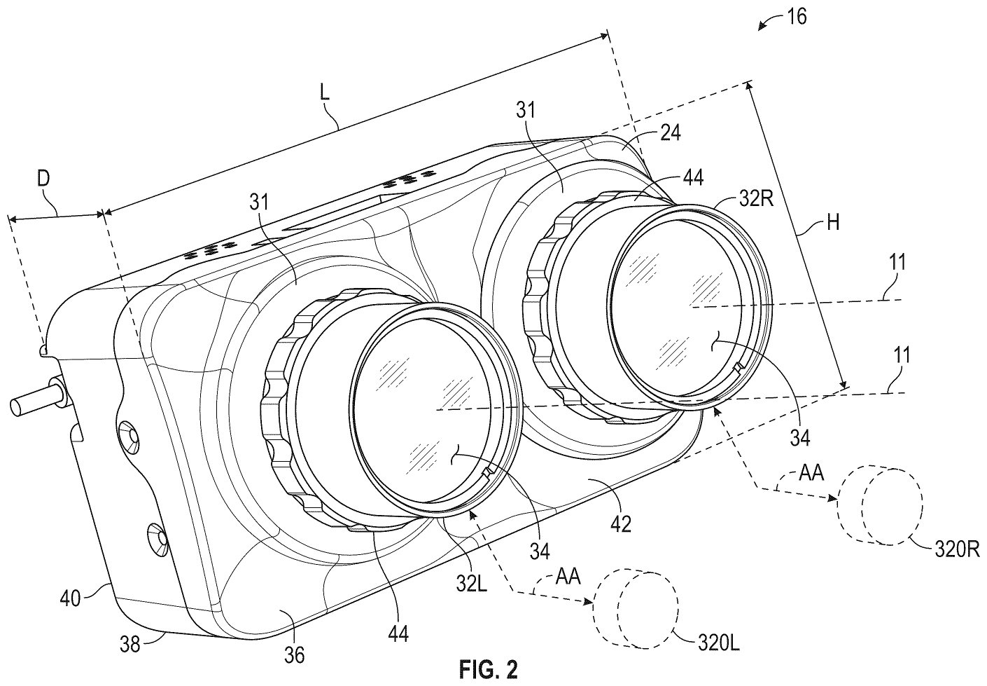

Referring to the drawings, wherein like reference numbers refer to like components, and beginning with FIG. 1, a visualization system 10 constructed in accordance with the present disclosure includes a mounting bracket 12 such as a C-mount, a microscope 14 coupled to the mounting bracket 12, and digital binoculars 16. As set forth in detail below with reference to FIGS. 2-4, the digital binoculars 16 contemplated herein include multiple pairs of interchangeable eyepieces 32, one of which is visible in FIG. 1. The digital binoculars 16 include a housing 24 that surrounds and protects various internal components as set forth herein, including a pair of miniature display screens (“micro displays”) 25 for viewing displayed images of a patient's eye area (not shown). The housing 24 is shown in a non-limiting exemplary configuration, with other possible shapes, sizes, materials of construction, etc., being usable in other implementations.

The eyepieces 32 described herein are “interchangeable” in the sense that a clinician 22 using the microscope 14, e.g., an ophthalmic microscope, and the digital binoculars 16 may select from multiple different pairs of the eyepieces 32 without having to replace or change out the digital binoculars 16. Each available pair of eyepieces 32 has a corresponding eyepiece magnification level and object field-of-view (FOV), and is constructed with foreknowledge of the total magnification level of the visualization system 10 as a whole. In a possible approach, the clinician 22 during or before surgery may detach the existing eyepieces 32 having a first magnification level or object FOV, e.g., by unscrewing the eyepieces 32 from the digital binoculars 16. The clinician 22 may then attach another set of the eyepieces 32 having a second magnification level or object FOV to thereby change the magnification level/object FOV. This action may be taken without replacing the digital binoculars 16, as noted above. A calculation method for determining total magnification level enables the construction and use of the interchangeable eyepieces 32 as described below and facilitates widespread adoption of the digital binoculars 16 as an alternative viewing solution to heads up display (HUD) systems.

In the representative visualization system 10 of FIG. 1, the housing 24 is coupled to the microscope 14 via an articulated bracket 18. A digital camera 20 connected to a pair of image sensors 200 may be connected to the microscope 14 and configured as, e.g., a charge-coupled device (CCD), a complementary metal-oxide-semiconductor (CMOS), an electron-multiplying CCD (EMCCD), or another set of application-suitable digital image sensors configured to output high-resolution three-dimensional (3D) image data to the digital binoculars 16 for viewing by the clinician 22, for instance a surgeon or attending medical staff working within an ophthalmic surgical suite.

It is recognized herein that emerging digital binoculars based on virtual reality headsets come equipped with a specific set of eyepieces providing the digital binoculars with a fixed predetermined magnification level. That is, a user of a given set of digital binoculars is restricted to a single set of eyepieces. As a result, the user is required to use an entirely distinct set of digital binoculars when the user wishes to change magnification levels. This current technological limitation impedes widespread adoption of digital binoculars for ophthalmic surgeries and other uses.

The present approach addresses this need by accurately characterizing the total magnification of the microscope 14, the digital binoculars 16, and the multiple pairs of interchangeable eyepieces 32 as a collective whole. The disclosed approach also enables the digital binoculars 16 to accept the eyepieces 32 with different magnifications or fields of view, thus avoiding the need to swap out the digital binoculars 16 when different magnification levels/fields of view are desired. The present strategy may be used to optimize or choose the particular eyepieces 32 that account for factors of the microscope 14 such as its optical magnification and image sensor size/resolution, as well as the factors of digital binoculars 16 such as the size/resolution of the micro displays 25, object field of view (FOV), and visual acuity of the clinician 22, etc. A benefit of the present teachings is the provided ability to choose interchangeable eyepieces 32 with proper magnification and object FOV for comfortable viewing of the clinician 22 or/and better resolution when using the digital binoculars 16. Construction and use of the eyepieces 32 is predicated on full and accurate understanding of the magnification problem, with the method described below being directed to that task.

Referring to FIG. 2, an exemplary embodiment of the digital binoculars 16 includes a left eyepiece 32L and a right eyepiece 32R respectively situated in front of the left and right eye of the clinician 22 of FIG. 1 when the digital binoculars 16 are in use, thus forming a stereoscopic imaging system. The housing 24, which may be constructed of a lightweight but sufficiently rugged material and cleanable material such as aluminum or molded plastic, defines a housing cavity therewithin (not shown). The housing 24 may be a generally rectangular enclosure having a length (L), a height (H), and a depth (D). Optical lens assemblies of the eyepieces 32L and 32R may include a front lens 34 through which the clinician 22 views a target object, e.g., a patient's eye area (not shown) located along an optical axis of the microscope 14 of FIG. 1. The lens assemblies may include a complex lens with multiple elements, a single lens, and/or a Fresnel lens and be constructed of various application-suitable materials, including but not limited to glass or plastic, injected molded plastic, and/or molded glass, etc. To provide different magnification of images as contemplated herein, the clinician 22 of FIG. 1 would simply remove the eyepieces 32L and 32R and replace them with a distinct set, as represented in FIG. 2 by arrows AA and interchangeable eyepieces 320L and 320R.

Outer surfaces 36 of the housing 24 are arranged to form a generally rectangular shape as noted above, with lateral edges 38 extending between a rear surface 40 and a front surface 42. As used herein, “front” is the particular structure or surfaces located proximate the clinician 22. The eye pieces 32L and 32R thus extend toward the clinician 22 of FIG. 1 from the front surface 42 of the housing 24. In this particular embodiment, the left and right eye pieces 32L and 32R (together referred to as eye pieces 32) may be equipped with an outer ring 44 mounted to an annular base 31 surrounding a designated position such as a corresponding center axis 11.

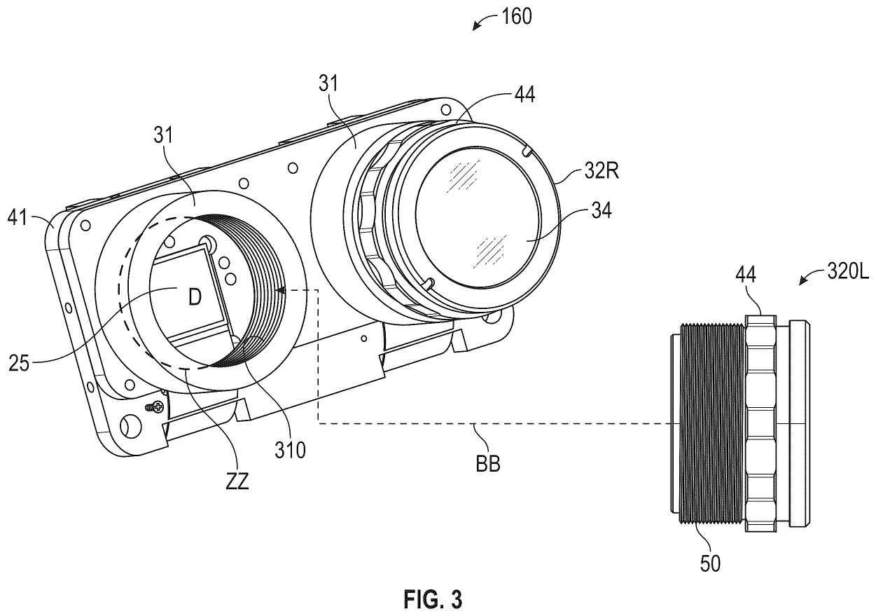

Referring to FIG. 3, an internal portion 160 of the digital binoculars 16 of FIG. 2 is shown with the left eyepiece 32L and housing 24 removed for illustrative clarity. The internal portion 160 may include a backplate 41 providing internal rigidity to the digital binoculars 16 of FIGS. 1 and 2. The front lens 34 is visible with the eyepiece 32R in place. As noted above, the digital binoculars 16 also include two micro displays (D) 25 located wholly inside of the housing 24 of FIG. 1 in a defined optical zone ZZ, with each respective one of the micro displays 25 being arranged on the corresponding center axis 11.

Each micro display 25 acts as a miniature television screen, e.g., a light-emitting diode (LED) screen, organic light-emitting diode (OLED) screen, or liquid crystal on silicon (LCoS) screen, the images on which are ultimately magnified or reduced by the interchangeable eyepieces 32 as needed. In operation, two separate images (left and right) are provided from the pair of image sensors 200 located in/on the microscope 14, e.g., on the digital camera 20 or sensors 200 that are integral with the microscope 14 of FIG. 1. While other image sensors 200 may be used, the microscope 14 of FIG. 1 may be equipped with the above-noted CMOS sensors in a possible non-limiting construction.

As noted above, the digital binoculars 16 described herein allow for use of the interchangeable/exchangeable eyepieces 32 with different magnification levels, which in turn could be optimized for the particular microscope 14 and/or clinician 22. In other words, the method described herein allows the clinician 22 to choose a set of eyepieces 32 having a desired magnification that is more suitable for or optimized to the clinician 22 and the particular microscope 14 in use. To that end, each annular base 31 may be configured to receive therein and securely engage the eyepieces 32.

Current thread designs are normally used for diopter adjustment, and thus are not suited to replacement of eyepieces 32 as in the present disclosure. A possible implementation includes constructing the annular bases 31 with an internally-threaded portion 310, for instance a representative thread pattern of M45×L9×P1.5−4 h. When a different eyepiece 32L is desired, the clinician 22 may unscrew the existing eyepiece 32L and replace it with another eyepiece 320L having a different magnification level or object FOV and an externally-threaded cylindrical portion 50 having a complementary external thread pattern, with replacement/interchangeability indicated by arrow AA.

That is, a thread type of the externally-threaded cylindrical portion 50 in one or more implementations is configured to directly engage a thread type of the internally-threaded portion 310 of the respective one of the annular bases 31. To better align the eyepieces 32, however, reliance on the thread itself may not be entirely sufficient, e.g., a precision alignment feature may be required. The clinician 22 would replace the eyepiece 32R in a similar manner. The magnification calculation method used herein ensures that the digital binoculars 16 may be constructed to accept multiple different eyepieces 32.

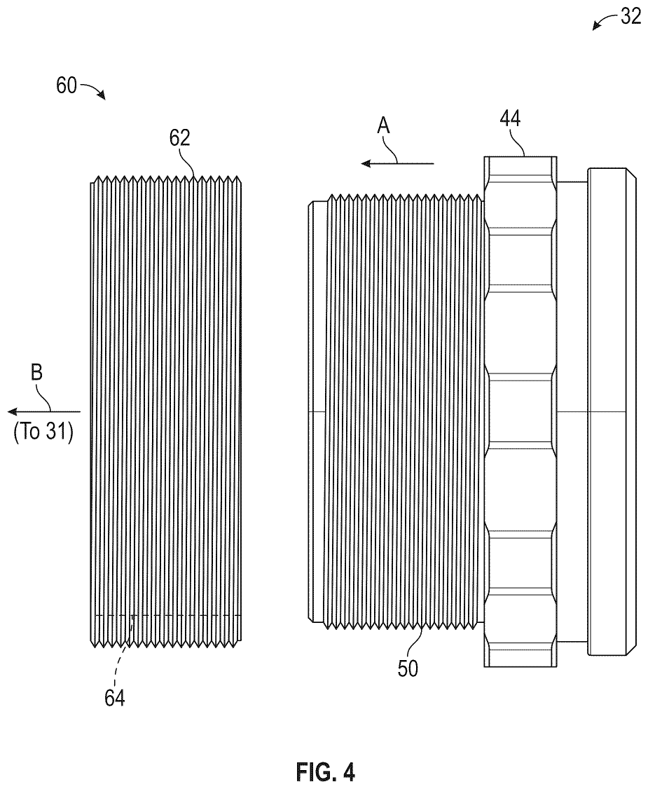

Referring briefly to FIG. 4, while it may be possible to directly thread the eyepieces 32 to the annular bases 31 as shown in FIG. 3, this is only possible when the internal thread pattern 310 complements the external thread pattern of the externally-threaded cylindrical portion 50. As noted above, reliance solely on threaded connections may not be optimally precise. In some embodiments, the external thread pattern of the eyepieces 32 may have a fixed thread pattern, i.e., one that corresponds to each the eyepiece 32 regardless of magnification level. In others, a separate annular adapter piece 60 may be used to mate up different thread types.

For instance, a first magnification level might have the above-noted M45×P1.5−4 h thread while a second magnification level has an M39×1.0 thread. That is, a thread type of the externally-threaded cylindrical portion 50 and a thread type of the internally-threaded portion 131 of the respective one of the annular bases 31 may be different in some embodiments. In this case, the annular adapter piece 60 may be used to connect the eyepiece 32 to the annular base 31 (FIG. 3), as indicated by arrow B, with the adapter piece 60 configured to connect the thread type of the externally-threaded cylindrical portion 50 and the thread type of the internally-threaded portion 131 of the respective one of the annular bases 31.

In a possible construction, the adapter piece 60 may be a threaded ring or annulus having an external thread 62 that is complementary to the thread pattern 310. The adapter piece 60 in this construction also includes an internal thread 64 that is complementary to the thread pattern on the externally-threaded cylindrical portion 50 of the eyepiece 32. Multiple adapter pieces 60 may be used to accommodate additional eyepieces 32 of different magnification levels or object FOV characteristics.

### Magnification Calculation:

Construction of the interchangeable eyepieces 32 may proceed by fully considering the total magnification level of the visualization system 10 of FIG. 1 as a whole. This may be achieved by calculations based on the following assumptions: (1) Let Mₒ represent the optical magnification of imaging optics of the microscope 14 of FIG. 1. The optics may include Common Main Objective (CMO) and two identical zoom optics to form left and right (stereo) images on two image sensors 200 via the camera 20; (2) the image sensors 200 are of a size H_{c}×V_{c} and pixels PH_{c}×PV_{c}, where H and V represent horizontal and vertical directions, respectively, and P indicates pixels; (3) Hₒ×Vₒ represents the object size (or object FOV) imaged to the image sensors 200; and (4) the micro displays (D) 25 of FIGS. 1 and 3 are of a size H_{d}×V_{d} and pixels PH_{d}×PV_{d} for the digital binoculars 16.

With respect to item (4), for the micro displays 25 to better display images from the image sensors 200 without losing image resolution, one of the following conditions should be satisfied:
1. Ideal Case: The micro display 25 and the image sensors 200 have the same aspect ratio for their respective horizontal and vertical sizes. Most commercially available image sensors have an aspect ratio of 16×9 (H×V). In addition, each micro display 25 should have an equal or greater number of pixels than the image sensors 200, i.e.:

$${H}_{c} / {V}_{c} = {H}_{d} / {V}_{d} ( = 16 / 9 ) and {PH}_{d} \geq {PH}_{c} and {PV}_{d} \geq {PV}_{c} \qquad ( 1 )$$

2. Likely Case: The micro displays 25 and the image sensors 200 have different aspect ratios, i.e., H_{c}/V_{c}≠H_{d}/V_{d}. Many commercial micro displays 25 are rectangular, with many of these being square. That is, H_{d}=V_{d} and PH_{d}=PV_{d}. Because the vertical FOV of the microscope 14 of FIG. 1 is more critical than its horizontal FOV, it should meet the following condition without losing image resolution:

$${PV}_{d} \geq {PV}_{c} \qquad ( 2 )$$

The image in the vertical direction of image sensors 200 should be fully displayed on the vertical direction of the micro displays 25. Issues may remain for the likely case due to the different aspect ratios of the image sensors 200 and micro displays 25 as discussed below.

Next, let ƒ be the effective focal length (EFL) of the eyepieces 32 for the digital binoculars 16. In this case, the optical magnification (M_{O}) of the microscope 14 may be calculated as:

$${M}_{o} = {H}_{o} / {H}_{o} = {V}_{o} / {V}_{o} \qquad ( 3 )$$

The optical magnification of eyepiece 32, i.e., ME, may be calculated as:

$${M}_{E} = 254 / f \qquad ( 4 )$$

where “254” is the normal viewing distance in millimeters (mm), and where f is likewise expressed in mm.

### Chip Magnification:

The magnification of the image on the image sensors 200 to the micro displays 25 (“chip magnification” M_{c}) may be generally expressed as:

$${M}_{c} = size of micro display / size of image sensor \qquad ( 5 )$$

Furthermore, one could calculate M_{c} for the assumption noted above that the aspect ratios of image sensors 200 and micro displays 25 are the same, as:

$${M}_{c} = {H}_{d} / {H}_{c} = {V}_{d} / {V}_{c} \qquad ( 6 a )$$

Magnifications along horizontal and vertical directions are the same when the image on the image sensors 200 is displayed on the micro displays 25 for the “ideal case” noted above.

For the “likely case” in which the aspect ratios of image sensors 200 and micro displays 25 are not the same—i.e., the aspect ratio of the image sensors 200 is usually with 16×9 while the micro displays 25 is a square—M_{c} can be calculated for the following situations:
(1) The image on the image sensors 200 along horizontal and vertical directions will be fully displayed on the micro displays 25. Then, the magnification along horizontal direction is:

$${M}_{C} H = {H}_{D} / {H}_{C} \qquad ( 6 b - 1 )$$

The magnification along vertical direction can be calculated as:

$${M}_{C} V = {V}_{D} / {V}_{C} \qquad ( 6 b - 2 )$$

M_{c}H≠M_{c}V, i.e., the magnifications along horizontal and vertical directions are not the same when the image on the image sensors 200 is displayed on the micro display 25. In this case the clinician 22 would see a distorted image on the micro displays 25.
(2) The image on the image sensors 200 along vertical directions will be fully displayed on the micro displays 25 while the image on the image sensors 200 along horizontal direction will be truncated and displayed on the micro displays 25 with the same magnification. Then we have:

$${M}_{c} = {M}_{C} H = {M}_{C} V = {V}_{D} / {V}_{C} \qquad ( 6 b - 3 )$$

The total magnification (M) of the microscope 14 can then be calculated as follows:

$$M = {M}_{O} * {M}_{C} * {M}_{E} \qquad ( 7 )$$

The total magnification M is thus equivalent to the total optical magnification from the microscope 14 to the eyepieces 32. The FOV (in degrees) of the digital binoculars 16 only depends on the eyepiece 32 and the micro displays 25, which can be calculated as:

$$FOVH = 2 * atan ( {H}_{D} / ( 2 * f ) ) \qquad ( 8 - 1 )$$

where FOVH is the FOV in the horizontal direction, and:

$$FOVV = 2 * atan ( {V}_{D} / ( 2 * f ) ) \qquad ( 8 - 2 )$$

for the FOVV is the FOV in the vertical direction.

For a micro displays 25 with a square shape, the FOVH&V in horizontal and vertical directions is stated as follows:

$$FOVH \& V = FOVH = FOVV = 2 * atan ( {H}_{D} / ( 2 * f ) ) = 2 * atan ( {V}_{D} / ( 2 * f ) ) \qquad ( 8 )$$

However, the clinician 22 may be accustomed to the FOV along the diagonal direction (Dd) of the micro displays 25 for the digital binoculars 16:

$$FOV = 2 * atan ( Dd / ( 2 * f ) ) = 2 * atan ( sqrt ( Hd 2 + Vd 2 ) / ( 2 * f ) ) \qquad ( 9 )$$

Selection of eyepieces for digital binoculars based on the FOV of Eq. (9) as set forth above does not properly include the factor of total magnification in Eq. (7). The present approach thus includes the factors in Eqs. (7) and (9) for the selection of the interchangeable eyepieces 32 for the digital binoculars 16 described herein. Using this approach, the eyepieces 32 may be optimized for the FOV of the digital binoculars 16 as well as the total magnification (or the resolution requirements) of the microscope 14.

### Interchangeable Eyepieces:

Optimal FOV of the digital binoculars 16 and how the particular resolution of the microscope 14 is represented thereon depends largely on the vision of the clinician 22. As a result, it is beneficial to provide multiple different pairs of eyepieces 32 with each pair having a different magnification or object FOV for the clinician 22 to meet different requirements and visions. Such an option is enabled herein to allow the digital binoculars 16 to be used with interchangeability of the eyepieces 32 of different magnifications.

A simplified illustrative example is that of a pair of digital binoculars 16 with at least two different pairs of eyepieces 32 and commercially-available micro-displays 25 with a rectangular or square shape. Thus, the clinician 22 could choose between hypothetical eyepieces 32, e.g., EP1 and EP2, based on the total magnification requirements and FOV to be perceived. Because digital binoculars 16 adopted from consumer virtual reality (VR) headsets only use one pair of eyepieces with fixed magnification, either EP1 or EP2 would come pre-mounted directly on the housing 24 via a predetermined mounting thread. The eyepieces EP1 and EP2 with their different magnifications would typically have different mounting threads, for instance M39×1.0 and M45×P1.5-4 h using the above non-limiting examples. Thus, a given housing 24 would threaded differently depending on the magnification of its mating eyepiece EP1 or EP2. In other words, absent the present teaching the eyepieces EP1 and EP2 would not be interchangeable on a given housing 24. The clinician 22 of FIG. 1 would therefore need two sets of digital binoculars 16 with two different mechanical housings 24 to accommodate eyepieces EP1 and EP2 in this example scenario.

The present calculations may be used during design and testing to develop the digital binoculars 16 with the interchangeable/exchangeable eyepieces 32. For example, the teachings set forth above may lend themselves to performance of a method for providing a total magnification level of the visualization system 10 of FIG. 1, i.e., one having the microscope 14, digital binoculars 16, and camera 20/image sensors 200. An embodiment of the method may include calculating the total magnification (M) of the visualization system 10 as a product of the optical magnification (M_{O}) of the microscope 14, the chip magnification (M_{C}) of the image sensors 200, and the optical magnification level (M_{E}) of first and second pairs of interchangeable eyepieces 32 having a corresponding magnification level and FOV.

The method may also include providing the digital binoculars 16 with connection structure to separately receive the first and second pairs of interchangeable eyepieces 32. The method may additionally include connecting the digital binoculars 16 to the microscope 14 and the image sensors 200 such that the visualization system 10 of FIG. 1 is provided with the total magnification (M) and the FOV when the first and second pairs of interchangeable eyepieces 32 are separately connected to the digital binoculars 16. As described above, the connection structure may include the pair of internally-threaded annular bases 31 connected to a housing 24 of the digital binoculars 16. Connection may be via a pair of the annular adapter pieces 60 (FIG. 4) each having (i) an externally-threaded surface 62 configured to engage a corresponding one of the internally-threaded annular bases 31, and (ii) an internally-threaded surface 64 configured to engage an externally-threaded cylindrical portion 50 of the first and second pair of eyepieces 32.

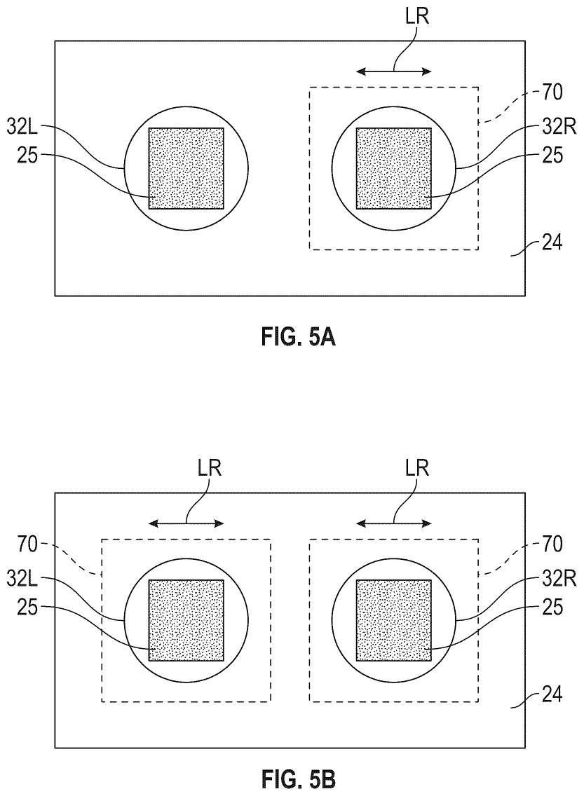

In one or more embodiments, the distance between the left and right eyepieces 32L and 32R may be adjusted based on the interpupillary (IPL) distance of the user, e.g., a moveable platform 70 as described herein may be configured to translate relative to the housing 24 to accommodate different interpupillary distances. Referring briefly to FIGS. 5A and 5B, for instance, the housing 24 may be optionally modified to accommodate the moveable platform 70. Shown schematically for illustrative simplicity, the left eyepiece 32L of FIG. 5A may be stationary relative to the housing 24 while the right eyepiece 32R is moveable (or vice versa). The moveable eyepiece, in this instance the right eyepiece 32R, may be couplable or connectable to the moveable platform 70. Both eyepieces 32L and 32R in this implementation would remain centered on a respective one of the micro displays 25 as described above. A user could thus translate the moveable platform(s) 70 relative to the housing 24 to position to the eyepieces 32L and 32R as close together or far apart as needed to accommodate the user's IPL. This may occur by translating the right eyepiece 32R of FIG. 5A closer to or farther away from the left eyepiece 32L as indicated by arrow LR, or by moving both eyepieces 32L and 32R in the same manner as shown in FIG. 5B.

Implementation of the movable platform(s) 70 of FIGS. 5A and 5B may be achieved in various ways within the scope of the disclosure. For example, the housing 24 may optionally define or include internal rails (not shown) spanning the length of the housing 24. The micro displays 25 may be couplable to the moving platform(s) 70 along with the eyepieces 32L and/or 32R, such that translation of the moveable platform(s) 70 carries the eyepieces 32L and/or 32R along with the micro displays 25. Locking features such as latches or detents could be used to arrest motion of the moving platform(s) 70 relative to the housing 24 when the latches/detents are released, thus maintaining the IPL distance for a given user.

Using the digital binoculars 16 of FIGS. 1-4, the clinician 22 of FIG. 1 would view the target object using both eyes in a relatively ergonomically friendly manner. This occurs without requiring polarized glasses, thus reducing the potential for disorientation or vertigo. Likewise, use of the digital binoculars 16 allows the clinician 22 to freely perceive the surrounding environment, for instance to locate surgical instruments or interact with operating room staff. These and other attendant benefits will be readily appreciated by those skilled in the art in view of the foregoing disclosure.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

Accordingly, such other embodiments fall within the framework of the scope of the appended claims. The detailed description and the drawings are supportive and descriptive of the disclosure, but the scope of the disclosure is defined solely by the claims. While various modes for carrying out the claimed disclosure have been described in detail, various alternative designs and embodiments exist for practicing the disclosure defined in the appended claims.

We claim:

1. A visualization system for use with an ophthalmic microscope having two image sensors, the visualization system comprising:
a first pair of interchangeable eyepieces;
at least one additional pair of interchangeable eyepieces, wherein each of the pair of interchangeable eyepieces and the at least one additional pair of interchangeable eyepieces has a corresponding magnification level and object field-of-view (FOV); and
digital binoculars in communication with the image sensors, the digital binoculars including:
a housing configured to connect to the ophthalmic microscope and defining a housing cavity;
a pair of annular bases connected to the housing, the annular bases being configured to separately engage the first pair of interchangeable eyepieces and the at least one additional pair of interchangeable eyepieces, and surrounding a corresponding center axis; and
a pair of micro displays positioned within the housing cavity.

2. The visualization system of claim 1, wherein each eyepiece of the first pair of interchangeable eyepieces and the at least one additional pair of interchangeable eyepieces includes an externally-threaded cylindrical portion configured to engage an internally-threaded portion of a respective one of the annular bases.

3. The visualization system of claim 2, wherein a thread type of the externally-threaded cylindrical portion is configured to directly engage a thread type of the internally-threaded portion of the respective one of the annular bases.

4. The visualization system of claim 2, wherein a thread type of the externally-threaded cylindrical portion and a thread type of the internally-threaded portion of the respective one of the annular bases are different, the visualization system further comprising:
an adapter piece configured to connect the thread type of the externally-threaded cylindrical portion and the thread type of the internally-threaded portion of the respective one of the annular bases.

5. The visualization system of claim 4, wherein the adapter piece includes an annular adapter piece having an externally-threaded surface configured to engage the internally-threaded portion of the respective one of the annular bases, and an internally-threaded surface configured to engage the externally-threaded cylindrical portion of the eyepieces.

6. The visualization system of claim 1, wherein the pair of micro displays include light-emitting diode (LED) screens, organic light-emitting diode (OLED) screens, or liquid crystal on silicon (LCoS) screens.

7. The visualization system of claim 1, wherein an aspect ratio of the micro displays and an aspect ratio of the image sensors are different.

8. The visualization system of claim 1, further comprising:
a moveable platform couplable to the first pair of interchangeable eyepieces and the at least one additional pair of interchangeable eyepieces, wherein the moveable platform is configured to translate relative to the housing to accommodate different interpupillary distances.

9. A visualization system, comprising:
a microscope;
a pair of image sensors connected to the microscope; and
a first pair of interchangeable eyepieces;
at least one additional pair of interchangeable eyepieces each having a corresponding magnification level and field-of-view (FOV); and
digital binoculars in communication with the image sensors, the digital binoculars including:
a housing connected to the microscope and defining a housing cavity;
a pair of annular bases connected to the housing, configured to engage the first pair of interchangeable eyepieces and the at least one additional pair of interchangeable eyepieces, and surrounding a corresponding center axis; and
a pair of micro displays positioned within the housing cavity.

10. The visualization system of claim 9, wherein the microscope is a digital microscope.

11. The visualization system of claim 9, wherein each eyepiece of the first pair of interchangeable eyepieces and the at least one additional pair of interchangeable eyepieces include an externally-threaded cylindrical portion configured to engage an internally-threaded portion of a respective one of the annular bases.

12. The visualization system of claim 11, wherein a thread type of the externally-threaded cylindrical portion is configured to directly engage a thread type of the internally-threaded portion of the respective one of the annular bases.

13. The visualization system of claim 12, wherein a thread type of the externally-threaded cylindrical portion and a thread type of the internally-threaded portion of the respective one of the annular bases are different, the system further comprising:
an adapter piece configured to connect the thread type of the externally-threaded cylindrical portion and the thread type of the internally-threaded portion of the respective one of the annular bases.

14. The visualization system of claim 13, wherein the adapter piece includes an annular adapter piece having an externally-threaded surface configured to engage the internally-threaded portion of the respective one of the annular bases, and an internally-threaded surface configured to engage the externally-threaded cylindrical portion of the eyepieces.

15. The visualization system of claim 9, wherein the pair of micro displays include light-emitting diode (LED) screens, organic light-emitting diode (OLED) screens, or liquid crystal on silicon (LCoS) screens.

16. The visualization system of claim 9, wherein the pair of micro displays are rectangular.

17. The visualization system of claim 9, wherein the micro displays and the image sensors have corresponding aspect ratios that are different from one another.

18. A method for providing a total magnification level of a visualization system having a microscope, digital binoculars with a pair of micro display screens, and a pair of image sensors, the method comprising:
calculating the total magnification of the visualization system as a product of an optical magnification (M_{O}) of the microscope, a chip magnification (M_{C}) of the image sensors to the micro display screens, and a magnification level (M_{E}) of a first pair of interchangeable eyepieces and at least one additional pair of interchangeable eyepieces each having a corresponding magnification level and object field-of-view (FOV); and
connecting the digital binoculars to the microscope and the image sensors such that the visualization system is provided with the total magnification and the object FOV when the first pair of interchangeable eyepieces and the at least one additional pair of interchangeable eyepieces are separately connected to the digital binoculars.

19. The method of claim 18, wherein connecting the digital binoculars to the microscope and the image sensors includes connecting a pair of internally-threaded annular bases of a housing of the digital binoculars to one of the first pair of interchangeable eyepieces and the at least one additional pair of interchangeable eyepieces.

20. The method of claim 19, wherein connecting the digital binoculars to the microscope and the image sensors includes connecting a pair of annular adapter pieces each having:
an externally-threaded surface configured to engage a corresponding one of the internally-threaded annular bases; and
an internally-threaded surface configured to engage an externally-threaded cylindrical portion of the first pair and multiple second pairs of eyepieces.
